(19) United States
(12) Patent Application Publication
(10) Pub. No.: US 2005/0020975 A1
(43) Pub. Date: Jan. 27, 2005

(54) [Khan Orotracheal Suction System]

(75) Inventor: Iftikhar Khan, Royal Oak, MI (US)

Correspondence Address: Iftikhar Khan, 4000 W 13 Mile Road Unit 21, Royal Oak, MI, 48073, US

(73) Assignee: Dr. Iftikhar Khan, Royal Oak, MI (US)

(21) Appl. No.: 10/604,469

(22) Filed: Jul. 23, 2003

(51) Int. Cl. A61M 29/00

(52) U.S. Cl. 604/96.01

(57) ABSTRACT

This abstract describes our product which is in the class of suction catheter systems. Our product is specifically designed for treating acute airway obstruction. Our system comprises a reservoir which is attached on one end to the wall suction vacuum using standard sump tubing. The other end is attached, via an adapter, to 15French suction tubing. This tubing is hooked to the suction catheters which the healthcare provider passes through an endotracheal tube into the trachea. A proximal balloon port is inflated with a 10 cc air filled syringe which inflates a distal cuff on the catheter. The distal cuff, which is 5 mm from the end of the catheter, creates a distal seal in the trachea which allows for the targeted suction force to remove the obstruction.

### BACKGROUND OF INVENTION

Every year thousands of patients in the United States die from acute airway obstruction. Tens of thousands of others die or suffer significant morbidity from the after affects of improper suctioning early; including aspiration pneumonia, empyema, and Acute Respiratory Distress Syndrome (ARDS). In June of 2003, I treated a patient who had aspirated twice at a large Level I trauma center. He sustained a sub-arachnoid hemorrhage from blunt trauma. There were no good oral suction catheters with large lumens that could adequately suction large food particles in his mouth and trachea. He had eaten a large Chinese food meal, and had spongy seafood material lodged in his trachea, thereby obstructing it. It was very difficult to ventilate him, because some air could be inspired around the spongy material, but very little air could be expired out, since the food was acting like a one way valve. The suction catheters available did not have large enough lumens to suck out the foreign material and other commercial suction catheters that we tried did not have a good enough air seal deep in the trachea to suck these major food particles out. In addition to foreign bodies our system can be used for acute airway obstruction from mucous plugging which causes significant morbidity and mortality in patients with asthma and chronic obstructive pulmonary disease in emergency department and intensive care unit (ICU) settings. These patients also have difficulty ventilating on and off ventilators and consequences of these obstructions are frequently lethal.

### SUMMARY OF INVENTION

This invention is an orotracheal suction system which can be used in acute airway obstruction from foreign bodies, mucous plugging and for agressive suctioning of the trachea and bronchi after aspiration to help prevent aspiration pneumonia, empyema, and ARDS. The system can also be used for agressive suctioning of the oropharynx when large particles are present.

Even flexible bronchoscopy was tried, by a surgeon, in the emergency department on this patient under direct visualization, but it, unfortunately did not work. Again there was no distal seal for suctioning and the lumen of the suction apparatus was too small.

### BRIEF DESCRIPTION OF DRAWINGS

FIG. 1 below shows the system assembled with its key components

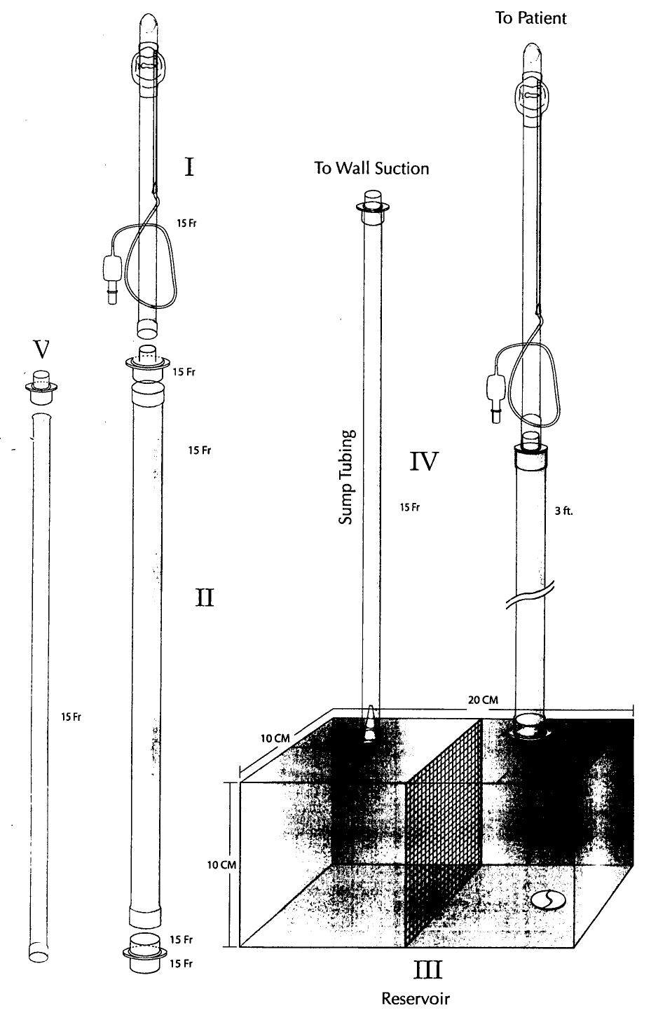

### DETAILED DESCRIPTION

The invention is an Emergent Orotracheal Suction System that could be attached to wall suction and have these key components. (1st component) A reservoir, measuring 2000 cc 20 cm×10 cm×10 cm, which on one end is attached to wall suction with standard sump tubing, and the other end is attached to our standardized extension tubing which measures 15French (Fr) in diameter. On the top of the 20 cm×10 cm surface of the reservoir, there is a 2 cm diameter tapering “male” entry port which is centered at 5 cm and 5 cm from the edge. The exit is protected by a grid which measures 2 mm×2 mm over the opening which prevents obstruction of the vacuum by large particles. The reservoir would also be halved on the inside by a 4 mm×4 mm plastic grid, which would keep large particles preferentially on the entry side of the reservoir. On the bottom of the entry side is a 5 cm diameter removable disc to empty particle contents on the entry side and evacuate fluid from the entire reservoir, and to clean it. The opposite hole is a 15Fr diameter “female” entry port which accepts the 15 Fr extension tubing via an adaptor which importantly keeps the entry to the reservoir 15Fr and is centered at 5 cm and 5 cm from the center edge of that side. (2nd component) The 15 Fr extension tubing should measure 3 ft-5 ft to allow enough slack to reach a patient's head on the stretcher. The extension tubing can then be attached via an adapter to our orotracheal suction catheters. Different adapters would accompany each suction catheter size. One side of the adapter would always provide a seal to the 15 Fr extension tubing and the other side to the different size orotracheal suction catheters. The catheters could range in size in an adult system from 5Fr to 8Fr, in 0.5Fr increments. The pediatric catheters could range in size from 0.5Fr to 5Fr, in 0.5Fr increments. The catheters work like this. They would be made of a high quality plastic polymer and have enough strength to withstand the pressure of the vacuum and flexibility to pass through. A catheter sized 0.5 Fr below the size of the endotracheal (ET) tube could be passed down the ET tube into the trachea. A proximal balloon port which would hook up to a 10 cc syringe would be on each suction catheter and could inflate a distal balloon on the catheter. The balloon would be 5 mm from the end of the catheter. This would create a good seal in the trachea for suction like an ET tube creates for ventilation. If the food bolus is very proximal in the trachea our extension tubing could be attached directly to an ET tube with our adapters to suction into the reservoir. The key problems our system would solve is large enough extension tubing and suction catheters to allow adequate suctioning of larger and smaller food particles which were aspirated in the trachea or vomited into the mouth. The other advantage of the catheters is there larger size and the distal seal they can create in the trachea. Standard sump tubing and other commercially available suction catheters frequently get clogged because their lumens are too narrow for large particles, or they are applying suction in the trachea with no good air seal. These commercially available tracheal catheters do not provide a good distal seal in the trachea to allow for proper suctioning of large and small aspirated particles. This system does not an unreasonable amount of parts. The suction catheter and extension tubing can even fit onto standard suction containers and the catheter pulled out frequently and used like a narrow commercially available suction catheter. This system will revolutionize the treatment of aspiration and acute airway obstruction in Emergency Departments (ED) and even Intensive Care Units(ICUs), across the country and internationally.

This system will make a major impact on acute acute airway obstruction because these people cannot be suctioned effectively in the Emergency Department and frequently die. They also develop serious aspiration pneumonias and ARDS because particles are always left in the trachea from inadequate suction and eventually expire for this very reason.

Even flexible bronchoscopy was tried, by a surgeon, in the emergency department on this patient under direct visualization, but it, unfortunately did not work. Again there was no distal seal for suctioning and the lumen of the suction apparatus was too small to remove the foreign body.

1. An orotracheal suction system consisting of an orotracheal suction catheter, extension tubing, and a resorvior which which can be used for suctioning of the oropharynx and trachea. The system can be used with all components or the catheter and extension tubing can be used with standard suction canisters currently available.

2. We claim the the first component is an orotracheal suction catheter which can be made of any plastic, synthetic polymer or other suitable material. The catheter can measure 5Fr to 5Fr or other suitable sizes for adult sizes and 0.5 Fr to 4.5Fr or other suitable sizes for pediatric patients. A proximal balloon port which would hook up to a 10 cc syringe would be on each suction catheter and could inflate a distal balloon on the catheter. The balloon can be 5 mm or other suitable lengths from the end of the catheter. As shown in the figure the catheter has a patient end which is inserted in the trachea for suctioning and method described earlier.

3. We claim the 2nd component to be 15 Fr (or larger or smaller diameter) extension tubing should measure 3 ft-5 ft (or larger or smaller) to allow enough slack to reach a patient's head on the stretcher. The extension tubing can then be attached via any suitable adapters (male-female or other) to our orotracheal suction catheters. It may be made of any plastic, polymer or other suitable material. Different size adapters would accompany each suction catheter size. One side of the adapter would always provide a seal to the extension tubing and the other side to the different size orotracheal suction catheters.

4. We claim the third component: A reservoir, measuring 2000 cc (or larger or smaller) 20 cm×10 cm×10 cm (or larger or smaller dimensions), which on one end is attached to wall suction with standard sump tubing, and the other end is attached to our standardized extension tubing which measures 15French (or) or larger or smaller in diameter. The resorvoir components may be made of any plastic, synthetic polymer or other suitable material. On the top of the surface of the reservoir, there is a 2 cm (or larger or smaller) diameter tapering “male” entry port which is centered from the edge. The exit is protected by a grid which measures 2 mm×2 mm (or larger or smaller) over the opening which prevents obstruction of the vacuum by large particles. The reservoir would also be halved is a 5 cm (or larger or smaller) diameter removable disc to empty particle contents on the entry side and evacuate fluid from the entire reservoir, and to clean it. The opposite hole is a large diameter “female” entry port which accepts the extension tubing via an adaptor which importantly keeps the entry to the reservoir a large diameter and is centered from the edge of that side.

5. We claim the Khan Orotracheal Suction system can be used with the reservoir or hooked directly to standard commercially available suction containers.
